(19) United States
(12) Patent Application Publication
(10) Pub. No.: US 2022/0357327 A1
(43) Pub. Date: Nov. 10, 2022

(54) OPTIMAL RANGE OF SALIVARY MMP-9 CONCENTRATION FOR SCREENING RISK OF PERIODONTITIS, AND METHOD OF ITS APPLICATION FOR SCREENING RISK OF PERIODONTITIS

(71) Applicant: Seoul National University R&DB Foundation, Seoul (KR)

(72) Inventor: Hyun-Duck KIM, Seoul (KR)

(73) Assignee: Seoul National University R&DB Foundation, Seoul (KR)

(21) Appl. No.: 17/735,532

(22) Filed: May 3, 2022

(30) Foreign Application Priority Data
May 7, 2021 (KR) 10-2021-0059469

(51) Int. Cl. G01N 33/573

(52) U.S. Cl. CPC G01N 33/573; G01N 2800/18; G01N 2333/96494

(57) ABSTRACT

Disclosed herein are an optimal range of salivary MMP-9 (matrix metalloproteinase-9) protein concentrations which allows for screening the risk of periodontitis, a method of its application for screening the risk of periodontitis using this range, the methods each comprising the steps: of measuring a concentration of MMP-9 protein in saliva taken from a subject; and establishing an optimal range of salivary MMP-9 concentrations which allows for screening the risk of periodontitis. Utilizing saliva taken in a non-invasive manner from a subject, the methods make it convenient to screen periodontitis. In addition, the methods have the effect of quickly and accurate determining the risk of periodontitis by measuring a concentration of MMP-9 protein present in a trace amount in saliva and enable periodontitis to be easily determined in clinics and at home.

### BACKGROUND OF THE INVENTION

### 1. Field of the invention

The present disclosure relates to an optimal range of salivary MMP-9 concentrations which allows for screening the risk of periodontitis, a method of its application for screening the risk of periodontitis using the range.

### 2. Description of the Prior Art

Periodontitis, which is a major oral disease, is an infectious disease caused by breakdown of balance between periodontopathic inflammation and immune response. When aggravated, periodontitis may lead to tooth loss.

Periodontitis is associated with elevated systemic inflammation through a change in proteins, immunoglobulins, and pro-inflammatory mediators. A diagnosis may be made of periodontal disease on the basis of various clinical indicators such as probing depth (PD) and clinical attachment level (CAL). However, such diagnostic methods are insufficient to alarm of the risk of onset of periodontitis in advance. Particularly important is early diagnosis of periodontitis because it allows for preventing irreversible destruction of the periodontium, maintaining a certain level of the alveolar bone, which supports teeth.

Matrix metalloproteinases (MMPs) are enzymes treat play an important role in pathological tissue destruction as well as physiological development and tissue remodeling and as such, are implicated in the onset and progression of periodontitis. Based on assessment of the substrate specificity thereof, MMPs are grouped into collagenases, gelatinases, stromelysins, and the membrane-type MMPs. MMP-9, which is one of the important gelatinases, is thought to be involved in destroying gingival and alveolar bone tissues during the onset and progression of gingivitis and adult periodontitis (Kim et al. 2014).

In this regard, Korean Patent Number 10-2016-7025378 A describes a method for diagnosing periodontal disease, using an MMP-8 activation product from the oral cavity, but does not disclose the use of salivary MMP-9 at all.

Particularly, given a discriminant accuracy of less than 60% for periodontitis, diagnostic methods make a decision at a seriously error rate resulting in false determination as to a positive of danger and a negative and thus is difficult to use in practice from a technical point of view. A diagnostic method, if less than 75% in sensitivity to periodontitis, is poor in discriminant accuracy for a positive of danger and thus greatly reduces use thereof in practical applications, with a high false rate for risk evaluation.

Therefore, there is a need for the development of a measure and method for determining the risk of periodontitis with effective performance in terms of both discriminant accuracy and sensitivity.

### Related Art Document

Patent Literature

(Patent literature 1) Korean Patent Number 10-2016-7025378 A

### SUMMARY OF THE INVENTION

With the aim of solving the problems encountered in the related art, the present disclosure is to provide an optimal range of salivary MMP-9 protein concentrations which allows for quickly and accurately predicting and screening the risk of onset and progression of periodontitis.

In addition, the present disclosure is to provide a method for screening the risk of periodontitis and a method for providing information for screening periodontitis, both using the optimal range of salivary MMP-9 protein concentrations.

The present disclosure provides an optimal range of salivary MMP-9 (matrix metalloproteinase-9) protein concentrations which allows for screening the risk of periodontitis, a method of its application for screening the risk of periodontitis, and a method for providing information for screening per the methods each comprising the steps: of measuring a concentration of MMP-9 protein in saliva taken from a subject; and establishing an optimal range of salivary MMP-9 concentrations which allows for screening the risk of periodontitis, wherein the range is between 16.75 to 44.00 ng/ml (both inclusive).

### BRIEF DESCRIPTION OF THE DRAWINGS

FIG. 1 is a receiver operating characteristic curve illustrating an optimal range of salivary MMP-9 protein concentration.

### DETAILED DESCRIPTION OF THE EXEMPLARY EMBODIMENTS

The present disclosure pertains to a method screening the risk of periodontitis and a method for providing information for screening periodontitis, the methods both using salivary MMP-9 protein, and encompasses a use of salivary MMP-9 protein in screening periodontitis, a kit for screening periodontitis, a composition for diagnosing or screening periodontitis, and all of the working embodiments thereof.

To all of the methods for screening the risk of periodontitis using salivary MMP-9 protein, the method for providing information for screening periodontitis, using salivary MMP-9 protein, the use of salivary MMP-9 protein in screening periodontitis, a kit for screening periodontitis, and the composition for diagnosing or screening periodontitis according to the present disclosure, the characteristic content of the present disclosure as set forth in the section <Optimal Range of Salivary MMP-9 Protein Concentrations for Screening Risk of Periodontitis>, below, is applied in its entirety. Other contents may be applied without limitations thereto as long as they do not degrade the purpose and advantages of the present, disclosure.

In the present disclosure, the protein matrix metalloproteinase-9 not only plays a role in physiological processes and tissue remodeling, but also is involved in pathological tissue destruction. Matrix metalloproteinase-9 is usually abbreviated to and hereinafter referred to as MMP-9.

The sequence of MMP-9 protein is well known and thus can be obtained by chemical synthesis (Merrifield, J. Amer. Chem. Soc. 85:2149-2156, 1963) or a genetic recombination technique. For chemical synthesis, a polypeptide synthesis method well known in the art may be available. For example, the protein defined in Korean Patent Number 10-2017-0098338 falls within the scope of the present disclosure.

As used herein in context with. periodontitis, the term “sensitivity” refers to the percentage (%) of those who are identified to be positive in patients who suffer from periodontitis, the term “specificity” to the percentage (%) of those who are identified to be negative in healthy persons who do not have periodontitis, and “discriminant accuracy” to the average percentage (%) of a sum of the sensitivity and the specificity.

<Optimal Range of Salivary MMP-9 Protein Concentrations for Screening Risk of Periodontitis>

According to an aspect of the present disclosure, an optimal range of salivary MMP-9 protein concentrations which allows for quickly and accurately predicting, screening, and evaluating the onset and progression of periodontitis has been established through tests on a massive number of persons and is provided.

In the present disclosure, the stage in which the onset of periodontitis is being in progress as a result of breakdown of balance between periodontopathic inflammation and immune response evaluated through an antigen-antibody reaction for salivary MMP-9 protein of subjects. Based on this evaluation, an optimal range of salivary MMP-9 protein concentrations which allows for quickly and accurately predicting, screening, and evaluating the onset and progression of periodontitis was established. As shown in Table 1 and FIG. 1 obtained from tests on a massive number of persons, the optimal range of salivary MMP-9 concentrations is determined to be 16.75 to 44.00 ng/ml which can be selectively used according to purposes including the risk prediction, screening, and prognosis evaluation of periodontitis.

The establishment of an optimal concentration of salivary MMP -9 protein for determining the risk of periodontitis requires taking full account of the discriminant accuracy that encompasses the sensitivity (percentage of those who are identified to be positive in patients who suffer from periodontitis and the specificity (percentage of those who are identified to be negative in healthy persons who do not have periodontitis) at a specific reference concentration.

Low sensitivity to periodontitis may lead to the error that even patients with periodontitis are determined to be normal and lose an opportunity to be properly treated. For early diagnosis of periodontitis, therefore, sensitivity to periodontitis should be 75% or higher, preferably 80% or higher, and more preferably 85% or higher. A sensitivity of less than 75% is practically useless as it greatly decreases the accuracy of determining a subject at a high risk of periodontitis to be positive.

In addition, according to Blicher B et al. (Validation of self-reported periodontal disease: a systematic review. J Dent Res 84 (10): 881-890, 2005) , validity is evaluated to be high when a sum of sensitivity and specificity is 120% or more for periodontal disease, that is, a comprehensive discriminant accuracy is 60% or more. At a discriminant accuracy of less than 60%, erroneous decisions of false positive or negative for periodontitis risk are made frequently. Thus, such a low discriminant accuracy cannot be practically applied in terms of technical aspect.

In order to establish an optimal range of salivary MMP-9 protein concentrations which allows for quickly and accurately predicting, screening, and evaluating risk, onset, and progression of periodontitis, saliva was taken from 710 Korean men and women and measured for MMP-9 (matrix metalloproteinase-9) protein concentration. Periodontitis screening ability according to the measured salivary MMP-9 protein concentrations was applied to an ROC curve. In this regard, determination was made of a range of MMP-9 protein concentrations for sensitivity of 75% or higher and a discriminant accuracy of 60% or higher. According to the present disclosure, the optimal range of salivary MMP-9 protein concentrations is 16.75 to 44.00 ng/ml.

In an embodiment of the present disclosure, a cutoff value is set to range from 16.75 to 44.00 ng/ml, with a sensitivity of 75% or higher and a discriminant accuracy of 60% or higher and preferably with a sensitivity of 75 to 93% and a discriminant accuracy of 60 to 64%.

<Screening of Risk of Periodontitis>

A method for screening the risk of periodontitis according to the present disclosure comprises the steps of: measuring a concentration of MMP-9 (matrix metalloproteinase-9) protein in saliva taken from a subject; and determining the subject to be a periodontitis patient when the salivary MMP-9 protein concentration is within a cutoff value.

In an embodiment of the present disclosure, the cutoff value may be in the range of 16.75 to 44.00 ng/ml, with a sensitivity of 75% or higher and a discriminant accuracy of 60% or higher and preferably with a sensitivity of 75 to 93% and a discriminant accuracy of 60 to 64%.

In another embodiment of the present disclosure, the cutoff value may he in the range of 18.0 to 44.0 ng/ml, with a sensitivity of 75% or higher and a discriminant accuracy of 62% or higher.

In another embodiment of the present disclosure, the cutoff value may be in the range of 28.0 to 41.0 ng/ml, with a sensitivity of 77% or higher and a discriminant accuracy of 63% or higher.

In another embodiment of the present disclosure, the cutoff value may be in the range of 28.0, to 37.0 ng/ml, with a sensitivity of 80% or higher and a discriminant. accuracy of 63% or higher.

Furthermore, the method for screening periodontitis according to the present disclosure is characterized by measuring only MMP-9 protein concentrations in saliva to diagnose whether the subject is affected by periodontitis. That is, concentrations of salivary proteins other than MMP-9, such as MMP-8, MMP-13, IL-6, and IL-8, are not measured. As stated in the foregoing, periodontitis can be quickly and accurately screened and diagnosed by measuring only MMP-9 protein concentrations in saliva.

In the present disclosure, the saliva may be obtained in a non-invasive manner from a subject.

Available for the step of measuring a concentration of MMP-9 protein in saliva taken from a subject is any immunological method that known in the art, as exemplified by enzyme linked immunosorbent assay (ELISA), radioimmunoassay (RIA), sandwich ELISA, western blotting on polyacrylamide gel, immune-dot blotting assay, immunofluorescence assay (IFA), immunochemiluminescence assay, immunohistochemical staining, and immunochromatography (Rapid).

Conventionally, the diagnosis of periodontitis was not conveniently implemented because it utilized gingival crevicular fluid, which is taken in an invasive manner. Thus, conventional methods have difficulty in early diagnosing periodontitis.

In order to overcome the foregoing problems, the present disclosure is characterized by providing a method for screening periodontitis by using saliva taken in a non-invasive manner. Saliva can be readily taken in a non-invasive manner without aid of a skilled dental expert so that periodontitis can be easily screened in clinics and at home.

<Method for Providing Information for Screening Risk of Periodontitis>

A method for providing information for screening the risk of periodontitis according to the present disclosure comprises the steps of: measuring a concentration of MMP-9 protein in saliva taken from a subject; and determining the subject to be a periodontitis patient when the salivary MMP-9 protein concentration is a cutoff value or greater.

In an embodiment of the present disclosure, the cutoff value may be in the range of 16.75 to 44.00 ng/ml, with a sensitivity of 75% or higher and a discriminant accuracy of 60% or higher and preferably with a sensitivity of 75 to 93% and a discriminant accuracy of 60 to 64%.

In another embodiment of the present disclosure, the cutoff value may be in the range of 18.0 to 44.0 ng/ml, with a sensitivity of 75% or higher and a discriminant accuracy of 62% or higher.

In another embodiment of the present disclosure, the cutoff value may be in the range of 28.0 to 41.0 ng/ml, with a sensitivity of 77% or higher and a discriminant accuracy of 63% or higher.

In another embodiment of the present disclosure, the cutoff value may be in the range of 28.0 to 37.0 ng/ml, with a sensitivity of 80% or higher and a discriminant accuracy of 63% or higher.

Furthermore, the method for providing information for screening the risk of periodontitis according to the Present disclosure is characterized by using an optimal range of MMP-9 protein concentrations in saliva to provide information for screening the risk of periodontitis at home. That is, too high or low a concentration of MMP-9 protein in saliva is not employed and concentrations of salivary proteins other than MMP-9, such as MMP-8, MMP-13, IL-6, and IL-8, are not measured. As stated in the foregoing, information for screening periodontitis can be quickly and accurately provided by utilizing only an optimal range of MMP-9 protein concentrations in saliva.

In the present disclosure, the saliva may be obtained in a non-invasive manner from a subject.

Available for the step of measuring a concentration of MMP-9 protein in saliva taken from a subject is any immunological method that is known in the art, as exemplified by enzyme linked immunosorbent assay (ELISA), radioimmunoassay (RIA), sandwich ELISA, western blotting on polyacrylamide gel, immune-dot blotting assay, immunofluorescence assay (IFA), immunochemiluminescence assay, immunohistochemical staining, and immunochromatography (Rapid).

A better understanding of the present disclosure may be obtained via the following examples, which are set forth to illustrate, but are not to be construed to limit the present disclosure.

### EXAMPLES

1. Experiment Design and Ethical Consideration

University School of Dentistry reviewed and granted the ethical consideration for this study. All participants voluntarily provided an informed consent statement after hearing out full explanation of the purpose of the study. This is a cross-sectional study consisting of voluntary subjects recruited through advertisements targeting people living in the community. This study followed the Standards for Reporting of Diagnostic Accuracy Studies (STARD) guidelines.

2. Selection of participants

Criteria for selection of participants are as follows: 1) agreed to participate voluntarily; 2) no antibiotics administered in the last three months; 3) not pregnant; 4) agreed to provide sufficient saliva for this study.

Through this, 710 people aged 22-84 (188 negative periodontitis, 522 positive periodontitis; 307 males and 403 females) were recruited for the final analysis.

3. Assessment of Periodontitis

Periodontitis was classified according to the modification of new international classification of periodontitis (NICP) (Tonetti et al., 2018). Stages II-IV of NICP were accepted as periodontitis: 3 mm or more ABL, maximum probing depth (PD) of 4 mm or more, and radiographic hone loss of 15% or more.

Using the clinical attachment level (CAL) , which is a definitive measure of periodontitis, the mesial and distal pocket of the remaining teeth from the cemento-enamel junction (CEJ) of the tooth up to the deepest point of the tooth was assessed and recorded. All mesial and distal parts of the natural teeth, except the 3rd molars, were measured. Participants were dichotomized into participants with stage II-IV periodontitis (periodontitis positive) versus participants with no periodontitis or stage I periodontitis (periodontitis negative).

4. Saliva Sampling

Unstimulated whole saliva was collected from each participant after informing the participants on the sampling protocol. The participants were scheduled for saliva sampling at 8-12 in the morning time and were informed not to eat, drink, nor brush teeth 1 hour before sample collection. Collection of saliva was done using the passive drool or spitting method for 10 minutes in a 50 ml conical tube in order to maintain consistency of the collected samples. The secretion rate of saliva was recorded.

Vials containing saliva were centrifuged at 2600 g and 4° C for 15 minutes, followed by collection of the supernatant by 1 ml into autoclaved 1.5-ml Eppendorf tubes. The aliquot vials were then stored at −80° C until experiment, and the samples were thawed as needed for analysis.

5. Quantification of Salivary MMP-9 Protein

Concentrations of salivary MMP-9 protein were determined by a sensitive enzyme-linked immunosorbent assay (ELISA) kit.

Salivary MMP-9 protein was quantitated using a Quantikine® human MMP -9 immunoassay (R&D Systems, Inc., Minneapolis, Minn.) kit. All experiments were conducted according to the manufacturer's instruction.

6. Determination of Optimal Range of Salivary MMP-9 Concentration

In order to determine an optimal range of salivary MMP-9 concentrations for screening the risk of periodontitis, cutoff values of MMP-9 protein concentrations were established on the basis of the various measured concentrations of the salivary MMP-9 from the participants. Sensitivity, specificity, and discriminant accuracy were calculated for each cutoff value and assessed according to the following criteria, and the results are summarized Table 1, below.

### <Assessment Criteria>

| | Sensitivity | | |
|---|---|---|---|
| | | 75% (inclusive)− | |
| Discriminant Accuracy | ≥80% | 80% (exclusive) | <75% |
| ≥63% | ⊚ | ◯ | X |
| 60% (inclusive)- | ◯ | ◯ | X |
| 63% (exclusive) | | | |
| <60% | X | X | X |

TABLE 1

| MMP-9 | | | Discriminant | |
|---|---|---|---|---|
| Concentration | Sensitivity | Specificity | Accuracy | Assessment |
| (ng/ml) | (%) | (%) | (%) | Result |
| 1.03 | 99.8 | 5.3 | 52.6 | X |
| 3.92 | 98.3 | 12.2 | 55.2 | X |
| 16.75 | 92.9 | 27.1 | 60.0 | ◯ |
| 17.55 | 92.1 | 30.9 | 61.5 | ◯ |
| 18.48 | 91.4 | 33.0 | 62.2 | ◯ |
| 19.50 | 90.6 | 34.0 | 62.3 | ◯ |
| 20.15 | 90.0 | 34.6 | 62.3 | ◯ |
| 20.98 | 89.5 | 34.6 | 62.1 | ◯ |
| 22.45 | 88.7 | 36.2 | 62.5 | ◯ |
| 23.08 | 88.1 | 36.2 | 62.1 | ◯ |
| 25.01 | 86.8 | 37.2 | 62.0 | ◯ |
| 25.84 | 86.4 | 37.8 | 62.1 | ◯ |
| 27.89 | 85.1 | 39.4 | 62.3 | ◯ |
| 28.63 | 85.1 | 41.0 | 63.0 | ⊚ |
| 29.74 | 84.7 | 42.6 | 63.6 | ⊚ |
| 30.85 | 84.1 | 42.6 | 63.3 | ⊚ |
| 31.55 | 83.3 | 43.6 | 63.5 | ⊚ |
| 32.15 | 82.8 | 44.1 | 63.5 | ⊚ |
| 33.41 | 82.2 | 44.7 | 63.4 | ⊚ |
| 34.16 | 81.6 | 44.7 | 63.1 | ⊚ |
| 35.23 | 80.5 | 46.8 | 63.6 | ⊚ |
| 36.04 | 80.1 | 46.8 | 63.4 | ⊚ |
| 37.32 | 79.7 | 47.3 | 63.5 | ◯ |
| 38.11 | 79.1 | 47.3 | 63.2 | ◯ |
| 40.40 | 78.0 | 48.4 | 63.2 | ◯ |
| 40.99 | 77.6 | 48.4 | 63.0 | ◯ |
| 41.96 | 76.6 | 48.4 | 62.5 | ◯ |
| 42.81 | 75.5 | 48.9 | 62.2 | ◯ |
| 43.98 | 75.1 | 50.0 | 62.5 | ◯ |
| 60.26 | 64.2 | 58.0 | 61.1 | X |
| 69.52 | 62.1 | 61.7 | 61.9 | X |
| 80.00 | 59.6 | 64.4 | 62.0 | X |
| 100.05 | 55.0 | 70.2 | 62.6 | X |
| 200.14 | 33.9 | 90.4 | 62.2 | X |
| 302.49 | 27.0 | 95.2 | 61.1 | X |
| 400.08 | 21.1 | 96.3 | 58.7 | X |
| 606.24 | 14.2 | 98.9 | 56.6 | X |
| 806.38 | 11.3 | 98.9 | 55.1 | X |
| 1002.40 | 10.2 | 98.9 | 54.5 | X |
| 1205.96 | 7.7 | 98.9 | 53.3 | X |
| 1525.62 | 5.7 | 100.0 | 52.9 | X |
| 1989.40 | 3.8 | 100.0 | 51.9 | X |
| 2642.44 | 0.0 | 100.0 | 50.0 | X |

From the data in Table 1, it is understood that the sensitivity and the discriminant accuracy are 75% or higher and 60% or higher, respectively, at the cutoff of an MMP-9 protein concentration range from 16.75 to 44.00 ng/ml and the high sensitivity and discriminant accuracy desired by the present disclosure cannot be obtained at high or low concentrations beyond the range.

In greater detail, when the cutoff value of salivary MMP-9 protein concentrations was set to be less than 16.75 ng/ml, the discriminant accuracy was up to 55.2%, which was reduced by about 5%, compared to that at the cutoff value of 16.75 to 44.00 ng/ml. In Table 1, the discriminant accuracy has a minimum value of 50% and a maximum value of 63.6%. Considering that the deviation of the discriminant accuracy between minimum and minimum values is merely about it will he apparent to those skilled in that art that the difference of discriminant accuracy by 5% corresponds to a very large different in effect in the art.

In addition, when the cutoff value of salivary MMP-9 protein concentrations was set to exceed 44.00 ng/ml, the sensitivity was up to 64.2%, which was largely reduced by 10% or more, compared to that at the cutoff value of 16.75 to 44.00 ng/ml.

Therefore, the cutoff value of MMP-9 protein concentrations for screening the risk of periodontitis was determined to range from 16.75 to 44.00 ng/ml. A cutoff value out of the range significantly decreases the sensitivity and discriminant accuracy for the risk of periodontists and makes it difficult to accurately discriminate subjects at a high risk of periodontitis, obviously leading to a great loss in health science, both socially and economically.

The method for screening the risk of periodontitis according to the present disclosure guarantees an excellent sensitivity which amounts to 75% or higher for periodontitis patients, with a discriminant accuracy of 60% achieved comprehensively. Thus, the method meets the international standard suggested by Blicher et al. Guaranteeing the sensitivity and comprehensive discriminant accuracy that satisfy the international standard, the optimal range of salivary MMP-9 concentrations is the very good outcome first set forth in the world, which is very difficult to set forth in conventional manners.

As described hitherto, utilizing saliva taken in a non-invasive manner from a subject, the method for screening the risk of periodontitis and the method for providing information for screening periodontitis according to the present disclosure make it convenient to screen periodontitis. In addition, the methods have the effect of quickly and accurate determining the risk of periodontitis by measuring a concentration of MMP-9 protein present in a trace amount in saliva and enable periodontitis to be easily determined in clinics and even at home.

What is claimed is:

1. A method for screening a risk of periodontitis, the method comprising the steps of:
measuring a concentration of MMP-9 (matrix metalloproteinase-9) protein in saliva taken from a subject; and
establishing an optimal range of salivary MMP-9 concentrations which allows for screening the risk of periodontitis,
wherein the range is between 16.75 to 44.00 ng/ml (both inclusive). 2. The method of claim 1, wherein the saliva is unstimulated saliva. 3. The method of claim 1, wherein the screening of the risk of periodontitis is carried out in clinics or at home. 4. A method for providing information for screening a risk of periodontitis, the method comprising the steps of:
measuring a concentration of MMP-9 (matrix metalloproteinase-9) protein in saliva taken from a subject; and
establishing an optimal range of salivary MMP-9 concentrations which allows for screening the risk of periodontitis,
wherein the range is between 16.75 to 44.00 ng/ml (both inclusive).
